(19) United States
(12) Patent Application Publication
(10) Pub. No.: US 2009/0216278 A1
(43) Pub. Date: Aug. 27, 2009

(54) Method and device for stabilization

(75) Inventor: John K. Song, Chicago, IL (US)

Correspondence Address: TAYLOR & AUST, P.C., P.O. Box 560, 142. S Main Street, Avilla, IN, 46710, US

(73) Assignee: Dr. John K. Song, Chicago, IL (US)

(21) Appl. No.: 12/072,096

(22) Filed: Feb. 25, 2008

(51) Int. Cl. A61B 17/58

(52) U.S. Cl. 606/264; 606/279; 606/246

(57) ABSTRACT

A method and device for percutaneously mating a spinal connecting rod to spinal screws utilizing guide wires. Numerous embodiments are described whereby a connecting rod is passed over a guide wire through the skin and into the heads of spinal screws.

### FIELD OF THE INVENTION

The present invention relates to a method and device for percutaneously placing spinal stabilization instrumentation.

### BACKGROUND OF THE INVENTION

Complaints related to the spine make up a significant portion of annual visits to health care providers and lost productivity. Current treatments include both surgical and non-surgical means. Surgical treatments include common procedures such as laminectomy, discectomy, spinal fusions, and more recently disc and nucleus replacement. Non-surgical treatments include physical therapy, medications, and injections.

One of the most common problems of the spine is low back pain. It is unclear often times where the pain is coming from and what is causing it and unfortunately many patients simply have to suffer with this problem. For some, lumbar fusion procedures are an option.

A technique that has gained acceptance recently is the concept of minimally invasive spinal surgery. This involves conducting spinal surgery to do the least amount of damage to surrounding tissue as possible. Typically, much smaller incisions are made compared to standard open procedures.

A number of minimally invasive means of placing spinal screws and rods exist in the marketplace to date. Yet all are relatively similar in their implementation and only differ in the means of passing the rod to connect two or more screws. A typical spinal construct is denoted in “levels” with the construct spanning a disc space to be stabilized. A one level stabilization or fusion typically consists of first placing 2 screws (one placed into each of the ipsilateral pedicles of the vertebra above and below the level to be stabilized) and then connecting a rod to each of these screws. This procedure can be repeated for the contralateral side of the spine.

A common problem with many of the minimally invasive screw systems is the complexity of the instrumentation needed and the difficulty in placing the rods. Screws for most systems are placed in an identical manner. Cannulated screws are typically placed over a guide wire into the pedicles of the vertebra from a posterior approach. These screws are attached to “screw extenders” which are typically tubes rigidly attached to the head of the screw which extend out of the skin and allow control of the screw and placement of the connecting rod.

Rod placement varies by manufacturer. By using the screw extenders, the rod may be placed either (1) using a fixed guiding mechanism which passes the connecting rod in an arc through the muscle tissue and through one screw and then into the next screw; (2) a freehand rod passage in a similar manner to (1); and (3) sliding the connecting rod directly between the screw extenders which guides the rod down to the screws. The screw extenders are then removed once locking screws are placed.

It is the intent of the present invention to describe a novel method and device for allowing percutaneous spinal stabilization without specialized screw adapters, braces, or tubes, or using previously described rod placing techniques.

“Method and Device for Spinal Stabilization” (Song)
U.S. Patent Documents

| | | |
|---|---|---|
| 2338159 | January 1944 | Appleton |
| 2372866 | April 1945 | Tofflemire |
| 2697433 | December 1954 | Zehnder |
| 3892232 | July 1975 | Neufeld |
| 4335715 | June 1982 | Kirkley |
| 4409968 | October 1983 | Drummond |
| 4448191 | May 1984 | Rodnyansky et al. |
| 4545374 | October 1985 | Jacobson |
| 4573448 | March 1986 | Kambin |
| 4722331 | February 1988 | Fox |
| 4863430 | September 1989 | Klyce et al. |
| 4883048 | November 1989 | Purnell et al. |
| 4896661 | January 1990 | Bogert et al. |
| 4955885 | September 1990 | Meyers |
| 4957495 | September 1990 | Kluger |
| 5080662 | January 1992 | Paul |
| 5116344 | May 1992 | Sundqvist |
| 5163940 | November 1992 | Bourque et al. |
| 5171279 | December 1992 | Mathews |
| 5196013 | March 1993 | Harms et al. |
| 5242443 | September 1993 | Kambin |
| 5242444 | September 1993 | MacMillan |
| 5281223 | January 1994 | Ray |
| 5314429 | May 1994 | Goble |
| 5334205 | August 1994 | Cain |
| 5383454 | January 1995 | Bucholz |
| 5409488 | April 1995 | Ulrich |
| 5437667 | August 1995 | Papierski et al. |
| 5474551 | December 1995 | Finn et al. |
| 5569248 | October 1996 | Mathews |
| 5591165 | January 1997 | Jackson |
| 5591167 | January 1997 | Laurain et al. |
| 5601562 | February 1997 | Wolf et al. |
| 5613968 | March 1997 | Lin |
| 5613971 | March 1997 | Lower et al. |
| 5643273 | July 1997 | Clark |
| 5672175 | September 1997 | Martin |
| 5681320 | October 1997 | McGuire |
| 5683392 | November 1997 | Richelsoph et al. |
| 5704937 | January 1998 | Martin |
| 5720751 | February 1998 | Jackson |
| 5725532 | March 1998 | Shoemaker |
| 5735857 | April 1998 | Lane |
| 5741266 | April 1998 | Moran et al. |
| 5752962 | May 1998 | D'Urso |
| 5772594 | June 1998 | Barrick |
| 5851183 | December 1998 | Bucholz |
| 5871445 | February 1999 | Bucholz |
| 5891034 | April 1999 | Bucholz |
| 5891150 | April 1999 | Chan |
| 5891158 | April 1999 | Manwaring et al. |
| 5904683 | May 1999 | Pohndorf |
| RE36221 | June 1999 | Breard et al. |
| 5910141 | June 1999 | Morrison et al. |
| 5941885 | August 1999 | Jackson |
| 6036692 | March 2000 | Burel et al. |
| 6099528 | August 2000 | Saurat |
| 6123707 | September 2000 | Wagner |
| 6139549 | October 2000 | Keller |
| 6146386 | November 2000 | Blackman et al. |
| 6162223 | December 2000 | Orsak et al. |
| 6226548 | May 2001 | Foley et al. |
| 6235028 | May 2001 | Brumfield et al. |
| 6530926 | March 2003 | Davison |
| 6530929 | March 2003 | Justis et al. |
| 2002/0045904 | April 2002 | Fuss et al. |
| 2002/0161368 | October 2002 | Foley et al. |
| 2003/0229347 | December 2003 | Sherman et al. |
| 2005/0021031 | January 2005 | Foley et al. |

Foreign Patent Documents

| | | |
|---|---|---|
| 197 26 754 | June, 1997 | DE |
| 100 27 988 | January, 2002 | DE |
| 0839513 | June, 1981 | SU |
| WO 97/30666 | August, 1997 | WO |
| WO 97/38639 | October, 1997 | WO |
| WO 99/15097 | April, 1999 | WO |
| WO 99/26549 | June, 1999 | WO |
| WO 00/44288 | August, 2000 | WO |

### Other References

Sofamor Danek; The Spine Specialist, TSRH Pedicle Screw Spinal System, Severe Spondylolisthesis of L5-S1 Grade 3 & 4; Surgical Technique as described by Edward H. Simmons, MD, Edward D. Simmons, Jr. MD, Howard D. Markowitz, MD. .COPYRGT.1997. cited by other
Sofamor Danek, The Spine Specialist; Horizon Spinal System, Surgical Technique; as described by Samuel J. Laufer, M.D., J. Andrew Bowe, M.D. .COPYRGT.1999. cited by other
Posterior Percutaneous Spine Insturmentation; 9 Supp 1) Eur Spine J (2000) Accepted Sep. 4, 1999. cited by other.

### SUMMARY OF THE INVENTION

While the invention has been shown and described with reference to certain preferred embodiments, it will be understood by those skilled in the art that various changes and modifications in form and detail may be made therein without departing from the spirit and scope of the invention, as defined by the appended claims.

It is an objective of the present invention to present a simplified method of connecting a spinal connecting rod (hereafter called the “connecting rod”) to two or more spinal screws.

It is an objective of the present invention to present a connecting rod with features facilitating minimally invasive surgery.

It is an objective of the present invention to present a method of eliminating the complex equipment needed to perform minimally invasive spinal surgery.

It is an objective of the present invention to present a novel method of spinal stabilization by pre-assembling part or all of the final construct outside the body.

It is an objective of the present invention to present a novel method of spinal stabilization by altering the order in which the components are placed.

It is an objective of the present invention to present a means of changing the depth at which a spinal screw is placed while the connecting rod is in place.

It is an objective of the present invention to present a means of performing a simplified one or two level spinal fusion percutaneously.

It is an objective to describe a means of performing longer spinal fusions by passing guide wires from one skin incision under the skin and back out through a second skin incision to allow use of the novel device described in this body of work.

As an example only, with no intention of limitation of scope, the following is given as a possible sequence of events for a single level stabilization:
1) A disc space of interest is identified for the surgical procedure.
2) A cannulated trocar with central stylet is placed through the skin into a pedicle. The central stylet is removed
3) A guide wire is placed through the trocar into the vertebral body through the pedicle. The trocar is removed leaving the guide wire. This process is repeated for the next adjacent pedicle of interest on the ipsilateral side of the spine.
4) Both pedicles are prepared in the typical manner to accept a spinal screw and the screws are placed leaving the guide wires in place.
5) An appropriately sized connecting rod is selected and, using a novel feature to allow placing the guide wires through the rod, the rod is slid down the guide wires to the screws.
6) Locking screws are placed over the guide wires to secure the connecting rod to the spinal screws.
7) The guide wires are removed.

Alternatively, another embodiment of the present invention involves first placing one spinal screw, pre-assembling the second screw with the connecting rod outside the patient, and passing the screw-rod assembly down the guide wires and engaging the second screw into the bone until the instrumentation is in the appropriate position and mated with the first spinal screw. Locking screws can then be secured.

Alternatively, another embodiment of the present invention involves pre-assembling a single screw and a connecting rod outside the patient and placing these over the guide wires and engaging the screw into the bone. The connecting rod of this embodiment employs a modified feature to then allow the second screw to be placed over the wire into the bone and engaging the connecting rod. Then locking screws can be secured.

Alternatively, another embodiment of the present invention involves pre-assembling both spinal screws and the connecting rod together outside the patient and passing them both down to the spine over the guide wires and engaging the screws into their respective bones until appropriately positioned.

Any of the above embodiments can be construed to comprise a spinal connecting rod with slots or holes to allow guidance over a guide wire down to a spinal screw. Alternatively, simple rod adapters or collets with the necessary guide holes for the guide wires can be attached in some fashion to the spinal connecting rod thus eliminating the need for rod modification.

As one can see, the key features of the present invention are elimination of the screw extenders and placing the connecting rod, not using screw extenders, but by passing the guide wires through the rod down to the screws. Other benefits of the present invention allows screw adjustment with the connecting rod in place, pre-assembly of part or all of the instrumentation prior to implantation, and variability in the order in which components can be placed.

Spinal fixation devices is a generic term intended for any permanent implant which is secured to or placed into the spinal vertebral bone and can include hooks, clamps, wires, but most often it is a screw of sort form. Spinal fusion and spinal stabilization as used in this document are interchangeable and are intended to refer to traditional rigid rod placement or newer “soft” or “dynamic” stabilization techniques with flexible rods.

Guide wires, as used in this document, is used in the general sense as any device which can fulfill the roles described in the present invention and be conceived of by anyone skilled in the art in a reasonable manner. The device need only be made of bio-compatible material and the guide wire must pass in whole or in part through the device or instrument it is paired with. The guide wire need not be circular in cross-section or symmetric.

No limitation in the order of components placed or number of holes, screws, rods, or guide wires placed or number of levels operated on are intended by this body of work and where such is described, it is for illustration purposes only.

### BRIEF DESCRIPTION OF THE DRAWINGS

FIG. 1. Lateral view of two guide wires placed into stylized vertebral bodies through a stylized skin incision.

FIG. 2. Lateral view of a cannulated screw placed over a guide wire into a vertebral body through a skin incision.

FIG. 3. Isometric view of one embodiment of a spinal connecting rod with features for allowing passage of guide wires.

FIG. 4. Overhead and cross-sectional view of modified spinal connecting rod.

FIG. 5. Lateral view of a pre-assembled screw-rod construct being placed over guide wires to mate with a spinal screw already positioned in the vertebra.

FIG. 6. Final lateral view of construct from FIG. 5.

FIG. 7. Construct from FIG. 6 in place and a set screw being placed over the guide wire to secure the spinal connecting rod to the second screw.

FIG. 8(a,b) Lateral and isometric views of construct from FIG. 6 after guide wires are removed.

FIG. 9. Cross-sectional view of relationship of spinal instrumentation to the guide wire.

FIG. 10. Lateral view of preferred embodiment already in place in vertebral bodies.

FIG. 11. Construct from FIG. 10. illustrating both set screws being placed.

FIG. 12. Lateral view of alternate embodiment whereby an alternate pre-assembled screw-rod construct is mated with a traditional spinal screw.

FIG. 13. Isometric view of construct of FIG. 12.

FIG. 14. Lateral view of alternate embodiment where a the screw-rod assembly is placed first and a second screw is then mated to it.

FIG. 15. Isometric view of construct of FIG. 14.

FIG. 16. Lateral view of another alternate embodiment whereby the spinal screw protrudes through the spinal connecting rod.

FIG. 17. Isometric view of construct from FIG. 16.

FIG. 18. Isometric view of alternate embodiment where the spinal connecting rod is adapted to the guide wires by devices or features added to the rod.

FIG. 19. A lateral view of a construct requiring more than one skin incision to pass the guide wires.

FIG. 20. Construct of FIG. 19 where the guide wires from one skin incision are passed under the skin to exit through the second skin incision.

### DETAILED DESCRIPTION OF THE DRAWINGS

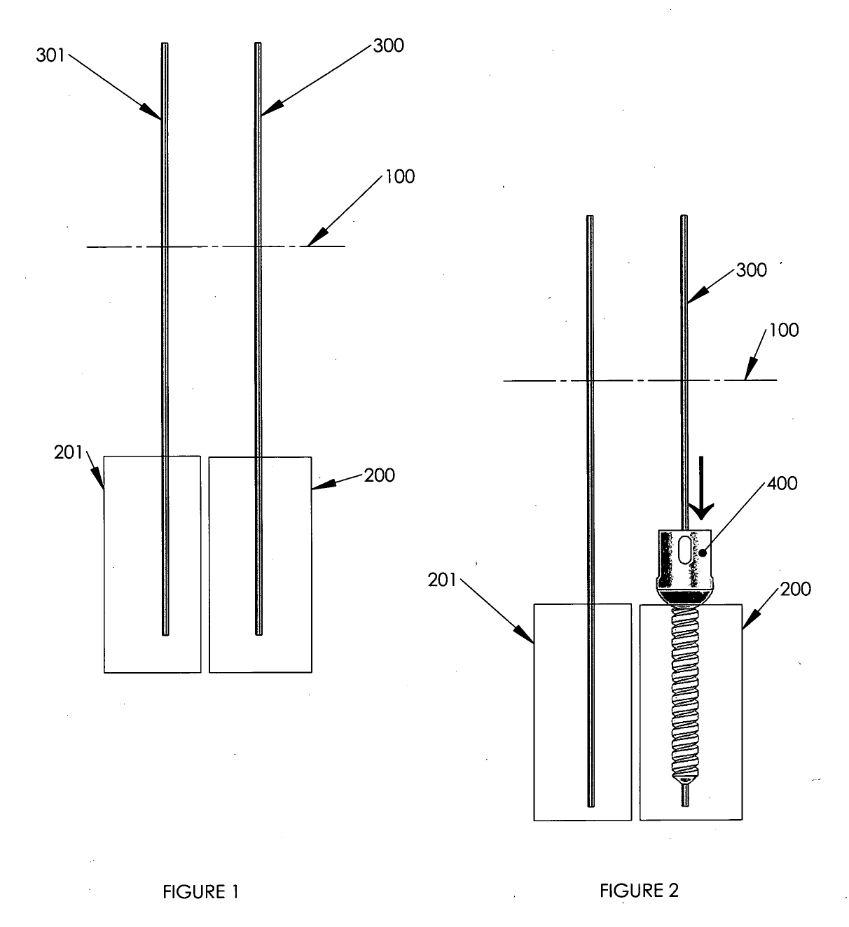

FIG. 1 illustrates two guide wires (300,301) placed through the skin (100) into two spinal vertebra (200,201).

FIG. 2 illustrates a typical spinal screw (400) placed over the guide wire (300) and passed through the skin (100) into the spinal vertebra (200).

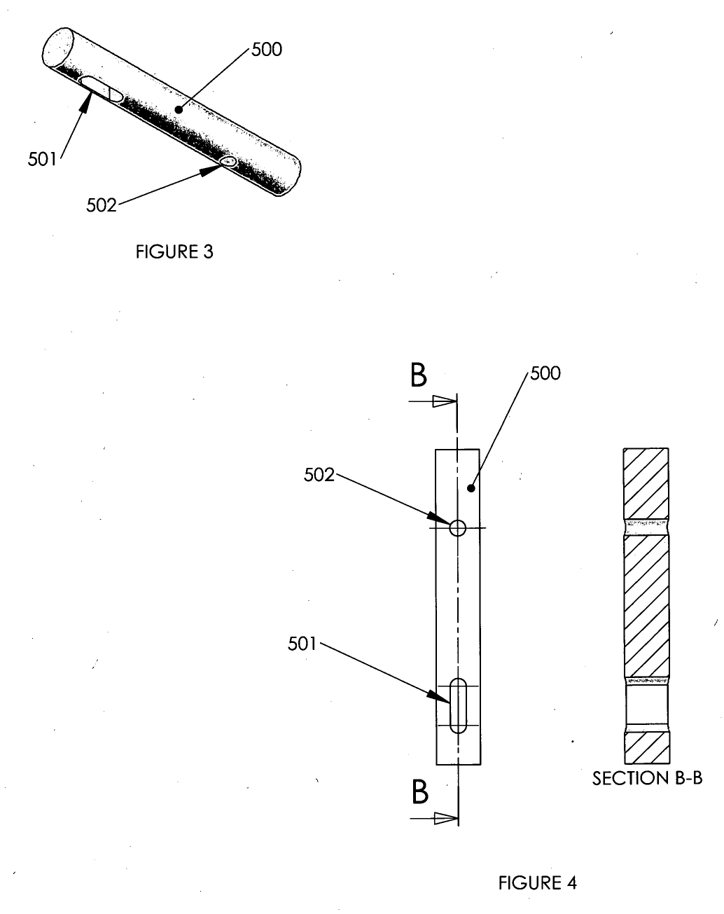

FIGS. 3 and 4 show the preferred embodiment of the device of the present invention. A spinal connecting rod (500) is modified by the creation of substantially transverse openings (501,502) in the rod. Note that the openings can be of any shape—depicted are a simple hole and a simple slot. These features may incorporate other features such as a bevel at the surface of the rod to allow the rod to be tilted more easily to facilitate passage through soft tissue. In addition, while the preferred embodiment is a closed opening (i.e. fully contained by the material of the connecting rod), it is conceivable that the openings can be offset laterally or longitudinally (to the left or right of section line B-B or at either end of the rod) and create an non-closed opening (i.e. not having material bounding it).

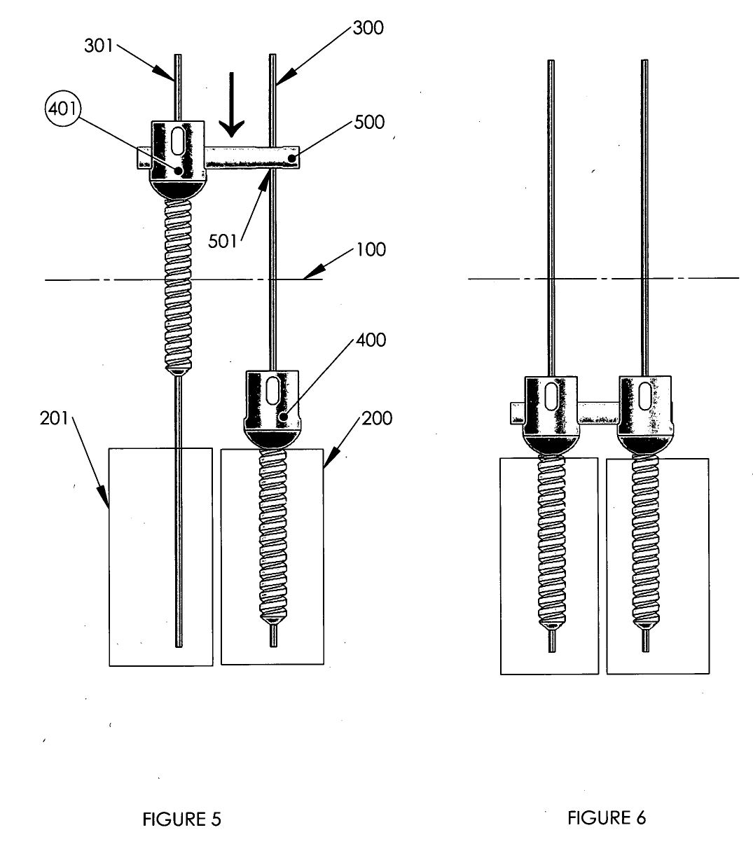

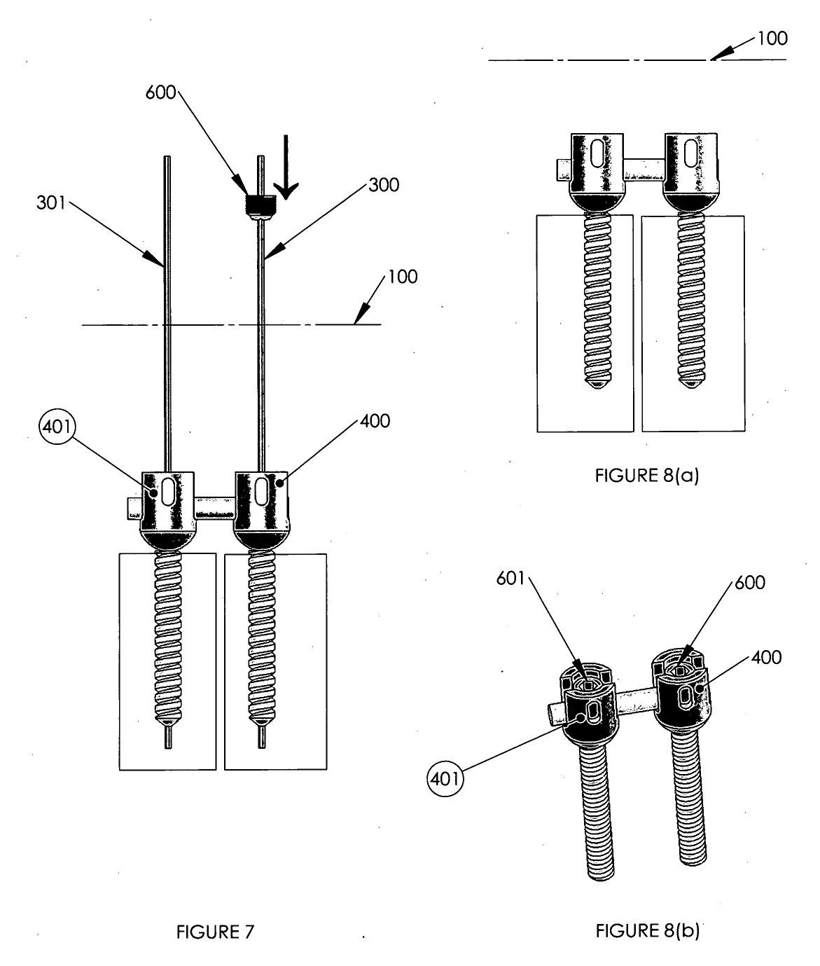

FIGS. 5-8 show one embodiment of the device of the present invention whereby a single level construct assembled. FIG. 5 shows the guides wires (300,301) placed into the vertebral bodies (200,201) through the skin (100). A first spinal screw has been placed over the guide wire (300) into the vertebra (200). A pre-assembled screw-rod contruct (401) is placed over the guide wires (300,301) outside the skin and passed through the skin. Rod (500) is mated to the first spinal screw (400). FIG. 7 shows a set screw (600) being passed over the guide wire (300) to bind the first spinal screw (400) to the rod (500). The screw of assembly (401) has a set screw (FIG. 8(b), 601) already in place.

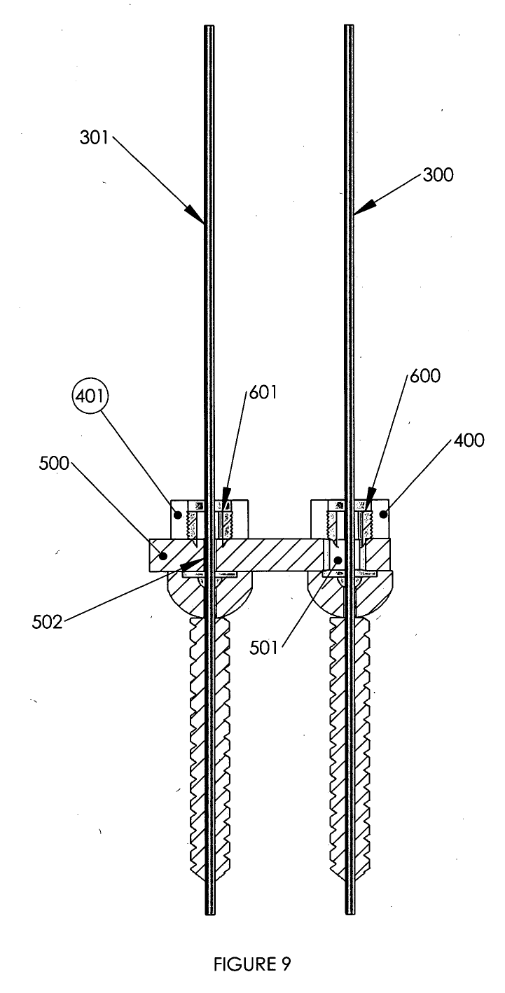

FIG. 9 is a cross-section showing the relative positions of the components used for the construct from FIGS. 5-8. Note that the rod is passed over the guide wire. This is markedly different from all other minimally invasive systems on the market today in which the rod is passed through guide tubes of sort in varying manners. Also note that while the features of the connecting rod (501,502) allow reliable guidance of the rod to the screw, its path is not constrained and the guide wires can be bent, the rod can be angled and shifted to allow the used to take any path through the soft tissue they desire. Also, the need for braces or other external adapters is eliminated. The rod from FIG. 3 has been used and guide wire 301 is passing through feature 502 of the connecting rod and guide wire 300 is passing through feature 501 of the connecting rod. Not depicted but assumed is that the screw-rod assembly (401) allows use of an instrument to place the screw of (401) into vertebral body (201).

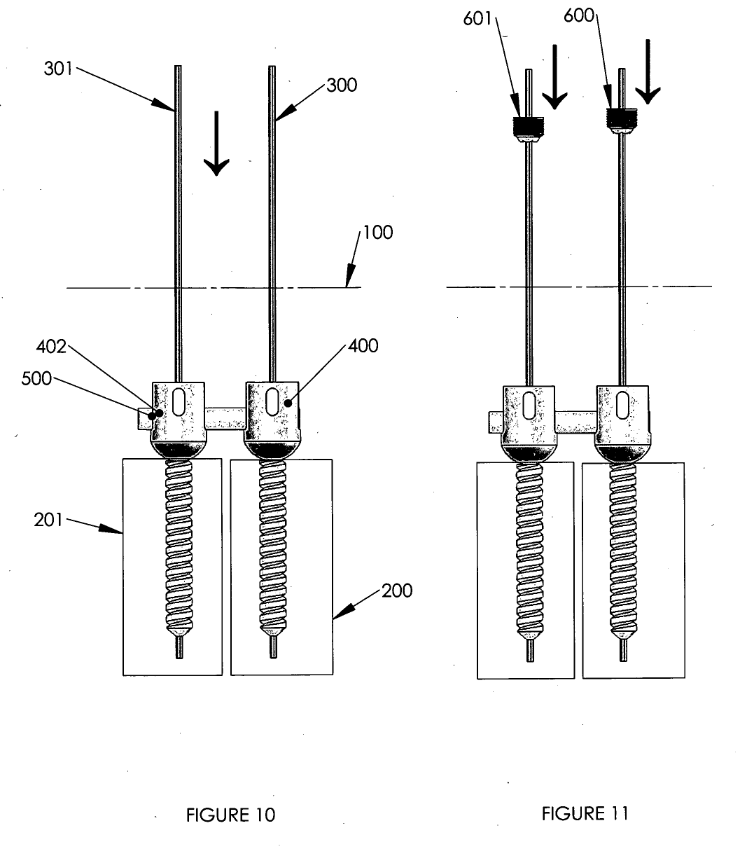

FIGS. 10 and 11 show the preferred embodiment with the screws (400,402) already placed over the guide wires (300,301) through the skin (100) into the vertebral bodies (200,201). The screws were first placed individually over each guide wire and then the rod (500) was placed over the guide wires into the screw heads. FIG. 11 shows the set screws (600,601) being placed over each guide wire to secure the rod to the screws.

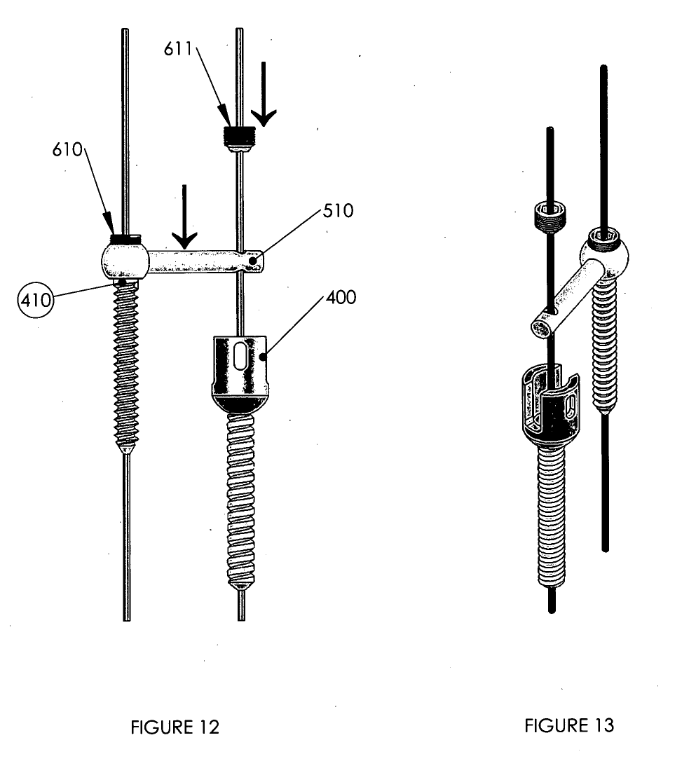

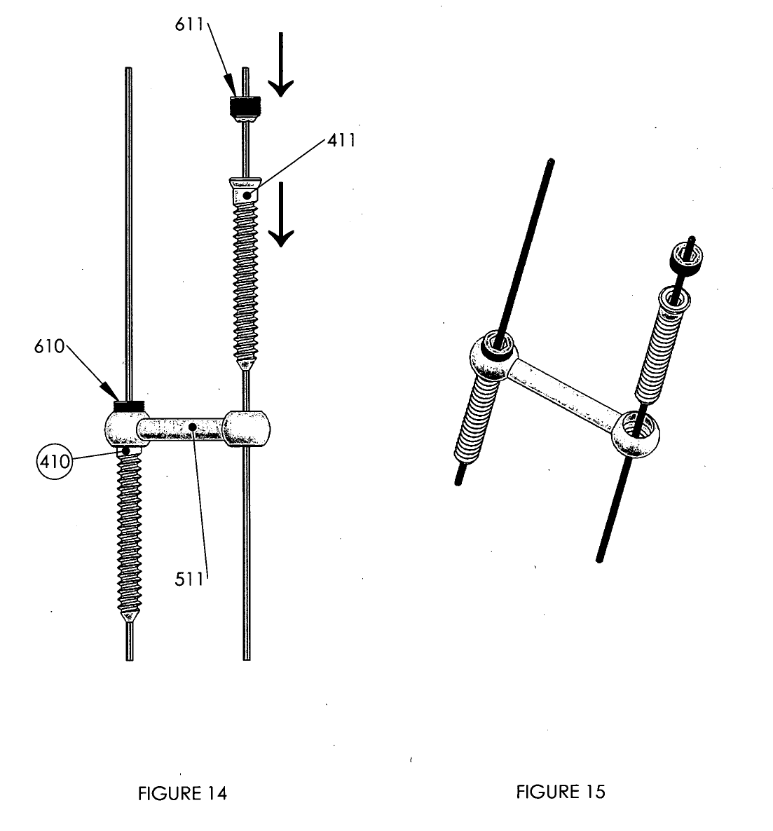

FIGS. 12-15 shows an alternate embodiment whereby a first spinal screw (400) is placed into the vertebral body through the skin over a guide wire. The stylized vertebra and skin have been omitted for clarity. A pre-assembled screw-rod construct (410) comprising a spinal screw placed through the rod (510) and secured by set screw (610) is passed over the guide wires in the manner previously described to mate with screw (400). Set screw (610) is placed to complete the assembly. FIG. 13 shows an isometric view of this same construct. While this particular construct does not seem much different from the embodiment depicted in FIG. 5, it is used to illustrate a different relationship of the components of the screw-rod construct (410). In the embodiment of FIG. 5, the rod of the screw-rod assembly was placed into the screw while in this embodiment, the screw of the screw-rod assembly is placed into the rod. Alternatively, the embodiment in FIG. 14 shows the screw-rod assembly (410) being placed first over the guide wires and the second screw (411) being placed over the guide wire (611) through the rod (511).

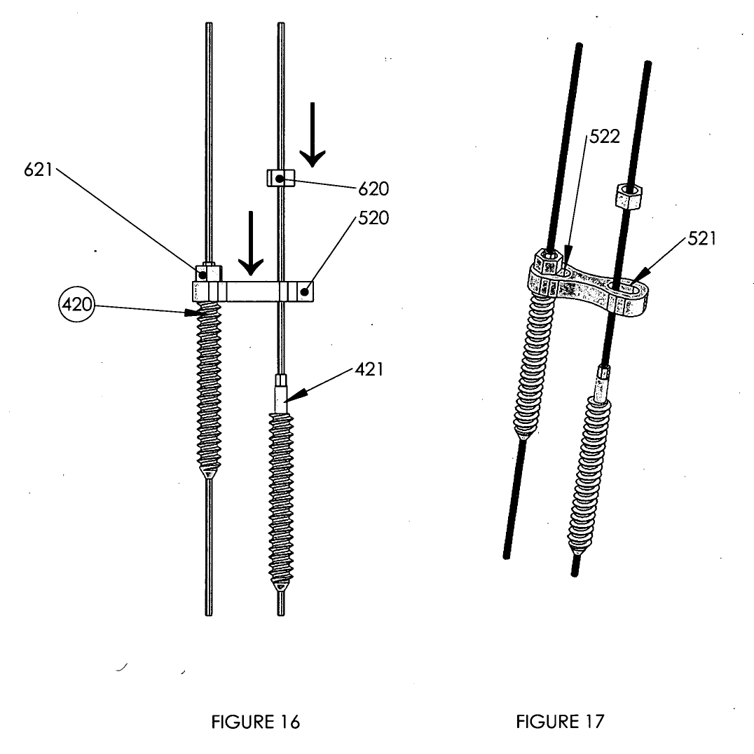

FIGS. 16 and 17 shows yet another embodiment whereby a pre-assembled screw-rod construct (420) is placed over the guide wires to mate with another screw (421). Set screw (621) was previously mated to the screw of assembly (42) to bind the connecting rod (520) to it. Set screw (620) is passed after assembly (420) to bind the rod (520) to screw (421). This shows a different relationship of the screw and the rod where the screw protrudes through the rod.

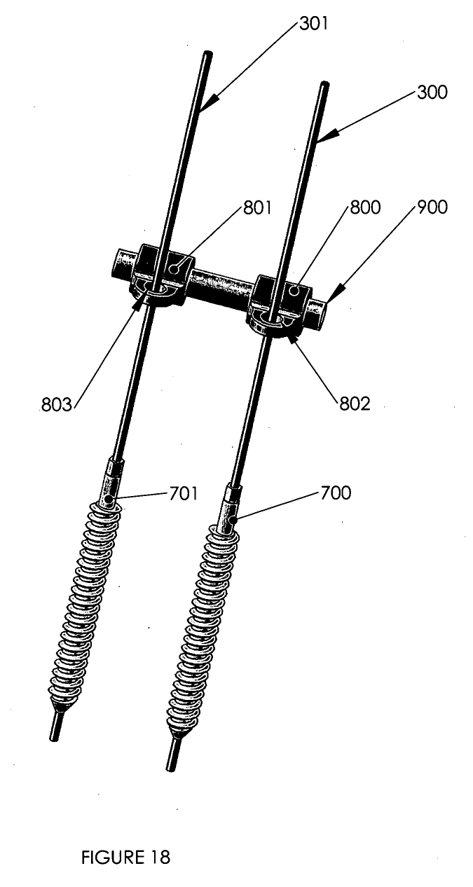

FIG. 18 shows an isometric view of an alternate embodiment of adapting the connecting rod to the guide wires. Shown is an isometric view of two screws (700,701) placed over two guide wires (300,301). Again, the skin and vertebral bodies have been omitted for clarity. A standard rod (900) without openings for guide wires is presented. Attached to the rod are adapters (800,801) which are mated with the rod (900) and have openings (802,803) for passage of a guide wire. The adapters can be integral parts of the rod (i.e. built in to the rod) or attached by common means to the rod in a separate process including welding, bonding, threading, retaining ring, set screw, etc.

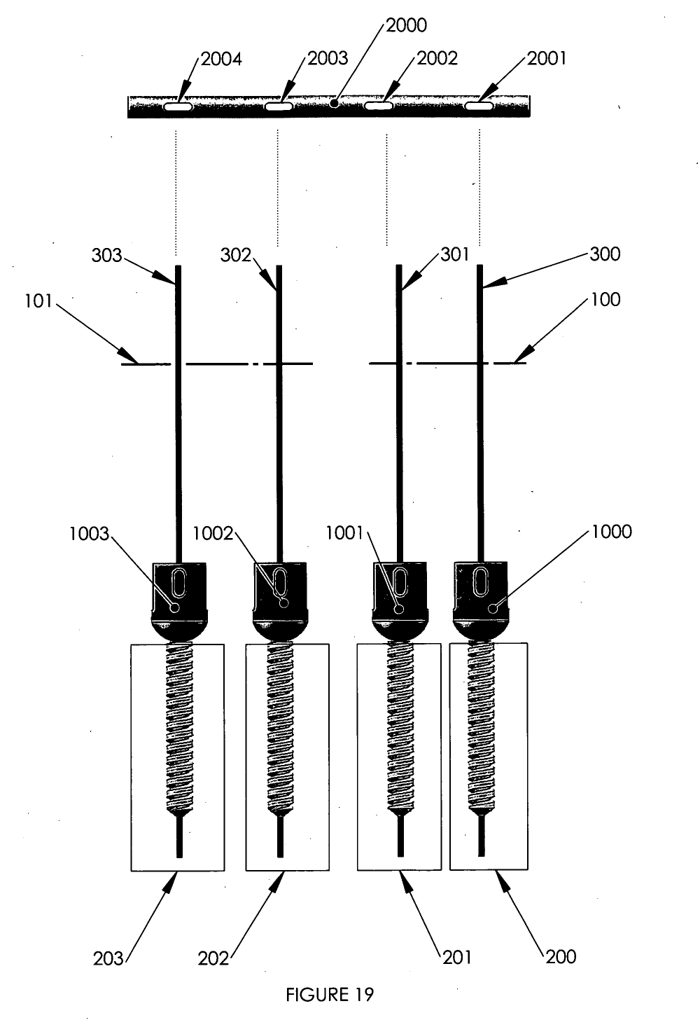

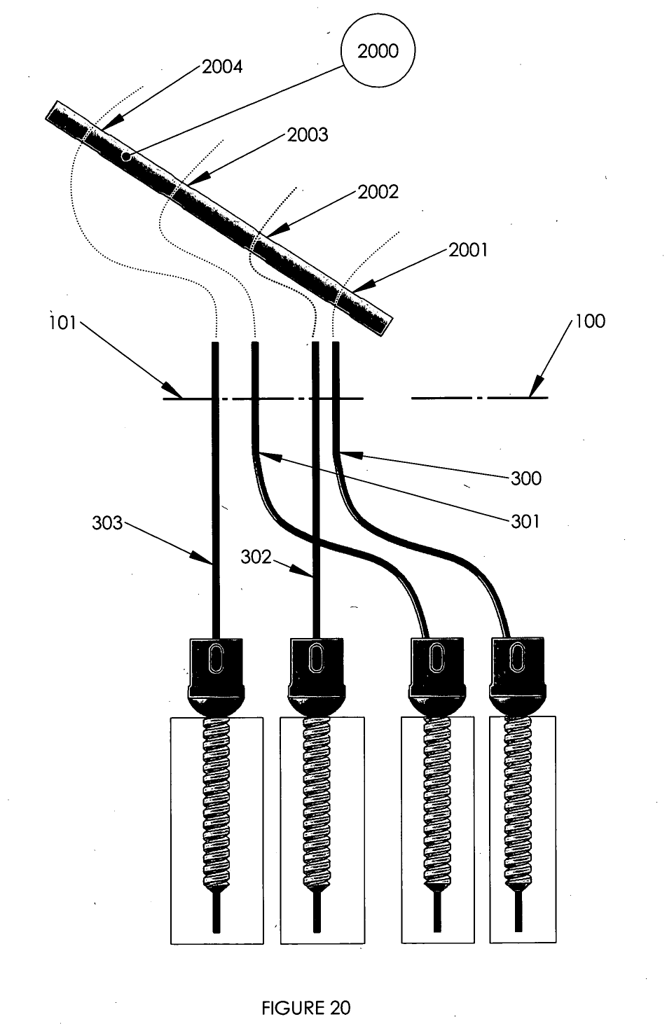

FIGS. 19 and 20 show an instance where guide wires can be passed from one skin incision to another to allow passage of a longer connecting rod. Guide wires (300,301) are passed through skin incision (100) into vertebral bodies (200,201) and guide wires (302,303) are passed through skin incision (101) into vertebral bodies (202,203). Spinal screws (1000-1003) are passed over each guide wire through their respective skin incisions and placed into the vertebral bodies. Connecting rod (2000) is pierced by openings (2001-2004) for the guide wires. Once the spinal screws are in place, the guide wires (300,301) from one skin incision (100) are passed under the skin to exit through the second skin incision (101). Subsequently, the connecting rod (2000) is passed over the guide wires using the respective guide holes (item 300 through feature 2001, item 301 through 2002, etc.) and the rod is angled to fit through skin incision (101) and then down into all of the screws.

What is claimed is:

1. A method of approximating a spinal connecting rod adjacent to spinal vertebra by placing one or more guide wires into the vertebra and then sliding the connecting rod over one or more guide wires to the spine.

2. A method of approximating a spinal connecting rod adjacent to spinal fixation devices by placing one or more guide wires into the vertebra, followed by placing one or more spinal fixation devices over one or more guide wires and fixating them in or to the vertebra, and then sliding the connecting rod over one or more guide wires to mate with one or more spinal fixation devices.

3. A method of performing spinal stabilization whereby the individual components are at least partially pre-assembled outside the patient and guided to the appropriate position over guide wires.

4. The method of claim 3 where one spinal screw is pre-assembled to one spinal rod and slid down a guide wire to mate with a second spinal screw.

5. The method of claim 3 where two spinal screws are pre-assembled with one spinal rod and slid down guide wires to mate with the spine.

6. A device for adapting a spinal connecting rod to mate with guide wires comprising a spinal connecting rod which has one or more features allowing one or more guide wires to pass through the connecting rod.

7. Device of claim 6 where the feature is a hole.

8. Device of claim 6 where the feature is a slot.

9. Device of claim 6 where the feature can also allow passage of part or all of the spinal fixation instrumentation.

10. Device of claim 6 where the feature can also allow passage of instruments for adjusting the spinal fixation instrumentation.

11. Device of claim 6 where the feature is a recess in the side of the connecting rod and open on one side.

12. Device of claim 6 where the feature has a bevel to facilitate angling the connecting rod easing passage through soft tissue.

13. A device for adapting a spinal connecting rod to mate with guide wires comprising a spinal connecting rod which has one or more openings oriented in a substantially transverse manner to the long axis of the rod used to pass guide wires.

14. A device for adapting a spinal connecting rod to mate with guide wires comprising a spinal connecting rod which has one or more openings oriented in a substantially longitudinal manner to the long axis of the rod used to pass guide wires.

15. A method of securing a spinal connecting rod to spinal fixation devices by placing a set screw over a guide wire and mating it with the spinal fixation device such that the fixation device and rod are subsequently bound together.

16. A method of securing a spinal connecting rod to spinal fixation devices by placing a set screw over a guide wire and mating it with the connecting rod such that the fixation devices and rod are subsequently bound together.

17. A device for securing a spinal connecting rod to spinal fixation devices comprising a set screw which has one or more openings used to pass a guide wire.

18. A device for adapting a spinal connecting rod to mate with guide wires comprising a spinal connecting rod and one or more rod adapters, said rod adapters comprising a means of fastening the rod adapter to the connecting rod and also comprising one or more openings oriented in a substantially transverse manner to the long axis of the rod which are used to pass guide wires.

19. A method of mating a spinal connecting rod to other spinal instrumentation whereby one or more guide wires are placed into one or more spinal vertebra, one or more spinal fixation devices are placed over the guide wires and mated with the spinal vertebra, and a spinal connecting rod placed over the guide wires and slid down to the spinal fixation devices and secured into place.

20. A method of mating a spinal connecting rod to spinal instrumentation whereby one or more guide wires are placed through a first skin incision, one or more guide wires are placed through a second skin incision, spinal fixation devices are placed over the guide wires into the vertebra, the guide wires from the first skin incision are tunneled under the skin to exit through the second skin incision, and a spinal connecting rod is slid over the guide wires through the second skin incision down to the spinal fixation devices.
